(19) United States
(12) Patent Application Publication
(10) Pub. No.: US 2006/0278225 A1
(43) Pub. Date: Dec. 14, 2006

(54) Delivery device

(75) Inventors: Donald Bruce Atherton MacMichael, Melbourn (GB); Douglas Ivan Jennings, Melbourn (GB); Jurgen Greiner-Perth, Radolfzell (DE); Lars Rune Gustaf Arvidsson, Lund (SE); Per Gustav Helgesson, Lund (SE)

Correspondence Address: FISH & RICHARDSON P.C., P.O BOX 1022, MINNEAPOLIS, MN, 55440-1022, US

(73) Assignee: AstraZeneca AB, a Sodertalje, Sweden corporation

(21) Appl. No.: 11/451,710

(22) Filed: Jun. 13, 2006

Related U.S. Application Data
(63) Continuation of application No. 10/250,537, filed on Nov. 4, 2003.

(30) Foreign Application Priority Data
Jan. 4, 2001 (SE) 0100034-8

(51) Int. Cl. A61M 16/00; A61M 11/00; A61M 15/00

(52) U.S. Cl. 128/205.16; 128/200.11; 128/200.14; 128/200.17; 128/203.22

(57) ABSTRACT

The present invention relates to a manually actuated delivery device for the delivery of a dose of liquid comprising a housing (1) which includes an outlet through which liquid, in use, is delivered. An actuating member (2) is moveably disposed relative to the housing (1) between a first position and a second position, the user moving the actuating member (2) into the second position to actuate the delivery device. A liquid delivery assembly (3) is located at least partly within the housing (1) the liquid delivery assembly (3) comprising a liquid delivery unit (4) a carrier unit (5) for the liquid delivery unit (4) and a coupling member (6) which couples the movement of the actuating member (2) and the carrier unit (5). The movement of the carrier unit (5) activates a counting mechanism (7a, 7b) which indicates how many doses have been delivered.

This application is a continuation (and claims the benefit of priority under 35 USC 120) of U.S. patent application Ser. No. 10/250,537, filed Jul. 2, 2003, which is the national phase application under 35 U.S.C. § 371 of PCT application Ser. No. PCT/GB01/05695, with an international filing date of Dec. 20, 2001, which claims priority to Swedish Patent application No. 0100034-8, with a filing date of Jan. 4, 2001, the contents of which are incorporated herein by reference.

The present invention relates to a manually actuated delivery device for the delivery of a volume of liquid, in particular, a liquid containing medicament.

WO 92/20455 discloses one example of such a delivery device which takes the form of a nasal inhaler for the delivery of a volume of liquid containing medicament as a spray into a nasal cavity. This delivery device includes a nosepiece in the form of an applicator portion for insertion into a nasal cavity. The applicator portion delivers the spray and is configured to be manually actuated by the application of a force axially relative to the longitudinal axis of the nosepiece.

Whilst such a delivery device is capable of delivering a metered volume of liquid, the use of the delivery device for delivery of a nasal spray can prove troublesome since the axial application of force tends to cause axial movement of the applicator portion within the nasal cavity which can lead to inefficient delivery of the liquid.

The present invention aims to overcome the problem of axial movement of the applicator portion by providing a delivery device configured such that the force for manual actuation is applied substantially orthogonally to the axis of delivery of the liquid. With such a configuration, inadvertent withdrawal or movement of the applicator portion can be obviated.

A further advantage of the present invention is the provision of a counting mechanism which indicates to the user how many doses of liquid have been delivered.

According to the present invention, there is provided a manually actuated delivery device for the delivery of a dose of liquid comprising a housing which includes an outlet through which liquid, in use, is delivered, an actuating member which is moveably disposed relative to the housing between a first position and a second position, the user moving the actuating member into the second position to actuate the delivery device and a liquid delivery assembly which is located at least partly within the housing, the liquid delivery assembly comprising a liquid delivery unit, a carrier unit for moving the liquid delivery unit along the longitudinal axis of the device and a coupling member which couples the movement of the actuating member and the carrier unit, characterised in that movement of the carrier unit along the longitudinal axis of the device activates a counting mechanism which indicates how many doses have been delivered.

Preferably, the carrier unit comprises at least one toothed ratchet finger which cooperates with a gear member of the counting mechanism.

Preferably, the gear member engages a toothed counting ring of the counting mechanism.

Preferably, the tooth pitch on the ratchet finger is constructed such that the gear member moves the toothed counting ring forward by one tooth for each dose delivered.

Preferably, anti-back drive means are provided to prevent the toothed counting ring rotating in a reverse direction.

Preferably, the anti-back drive means are located on the counting ring.

Preferably, the anti-back drive means are located on the internal surface of the housing.

Preferably, the anti-back drive means are in the form of a ratchet arm which engages the gear member.

Preferably, the gear member has an outer set of teeth which engage the toothed ratchet finger and an inner set of teeth which engage the toothed counting ring.

Preferably, the actuating member is a cover member which is moveably disposed between the first position in which the cover member at least partly covers the outlet and the second position in which the cover member acts as a lever which lifts the carrier unit.

Preferably, a seal is located within the cover member to seal the outlet when in the first position.

Preferably, the housing comprises an applicator portion with an outlet nozzle, the outlet nozzle being removable to enable cleaning.

Preferably, the outlet nozzle is threaded and cooperates with a threaded socket in the applicator base portion.

Preferably, the outlet nozzle “snaps” into the final position in the threaded socket.

According to a further aspect of the present invention, there is provided a manually actuated delivery device for delivery of a dose of liquid comprising a housing which includes an outlet through which liquid, in use, is delivered, and a cover member which his moveable between a first position in which the outlet is covered and a second position in which the outlet is exposed characterised in that a seal is located within the cover member to seal the outlet when in the first position.

Preferably, the cover member is hinged on the housing.

Preferably, the cover member is a snap-fit on the housing.

Preferably, the cover member has a screw-thread which cooperates with a screw-thread on the housing.

Preferred embodiments of the present invention will now be described in detail, by way of example only, with reference to the accompanying drawings, of which:

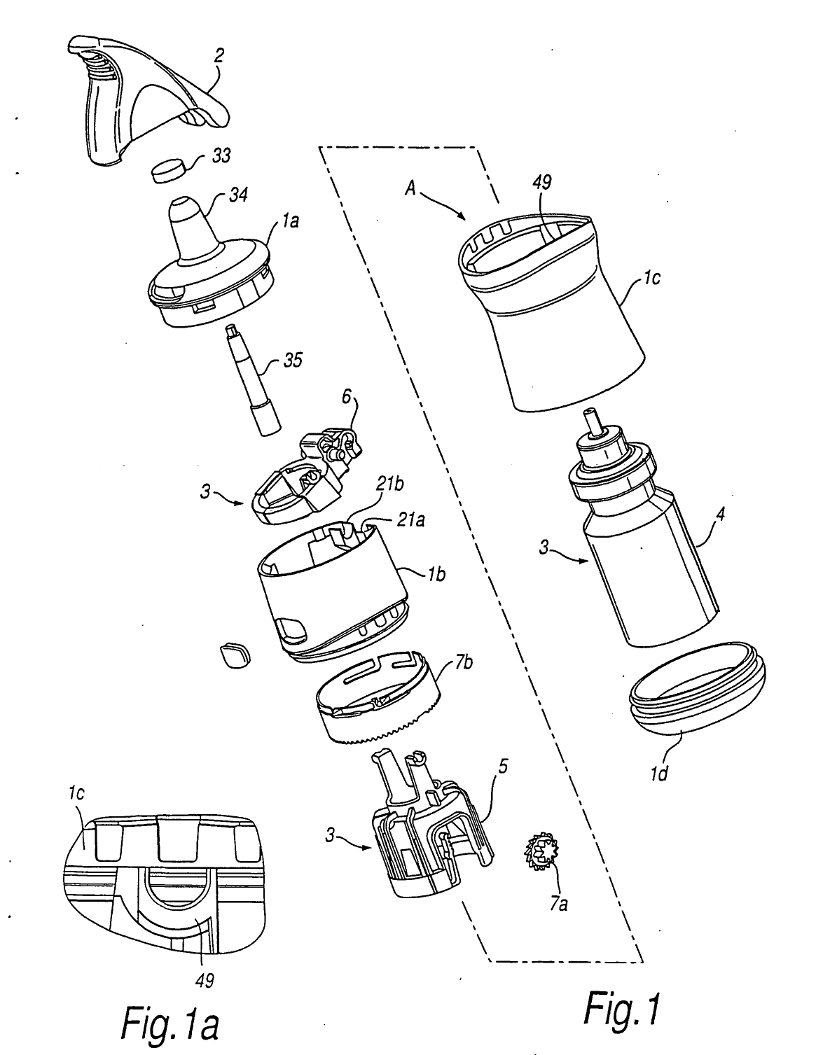

FIG. 1 is an exploded view from above a first embodiment of the delivery device;

FIG. 1a is an enlarged view in direction A in FIG. 1 of the upper part of the internal surface of the lower body portion;

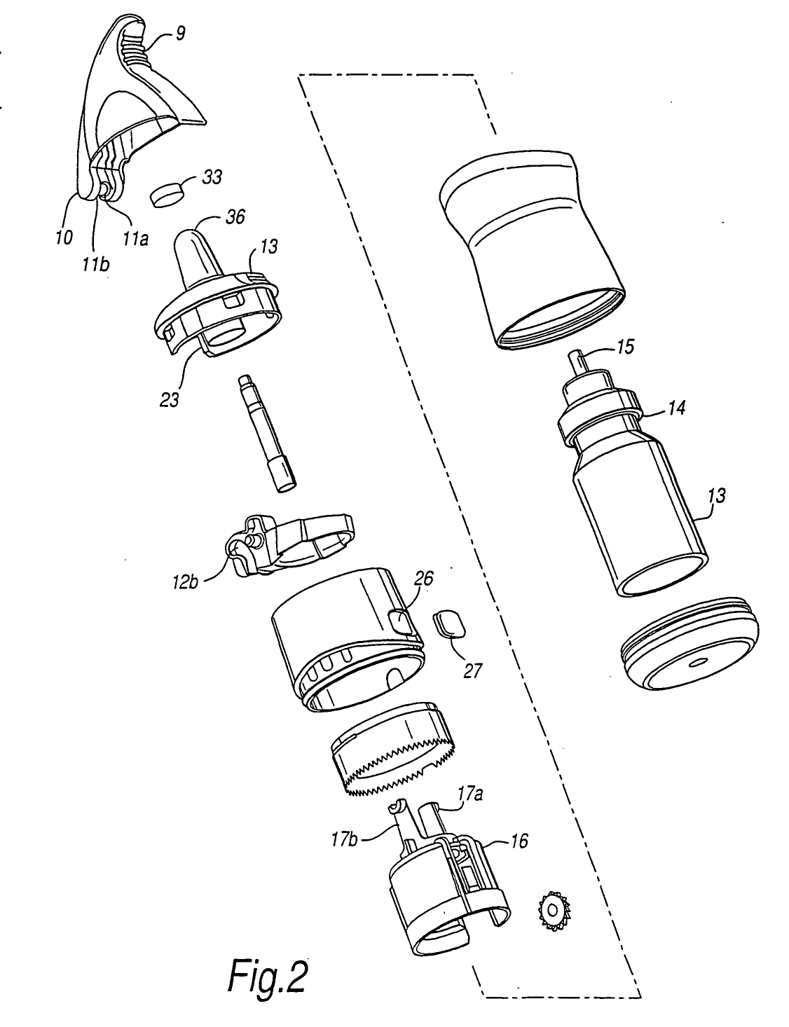

FIG. 2 is an exploded view from below of the first embodiment of the delivery device;

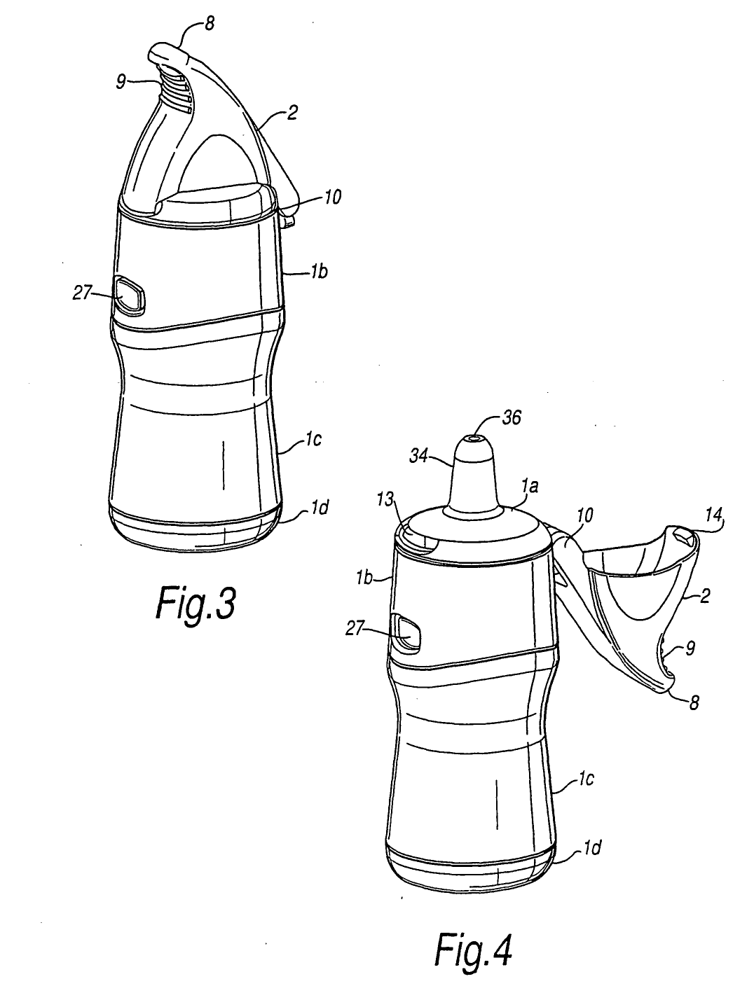

FIG. 3 is a perspective view of the assembled delivery device when closed;

FIG. 4 is a perspective view of the delivery device in FIG. 3 when opened;

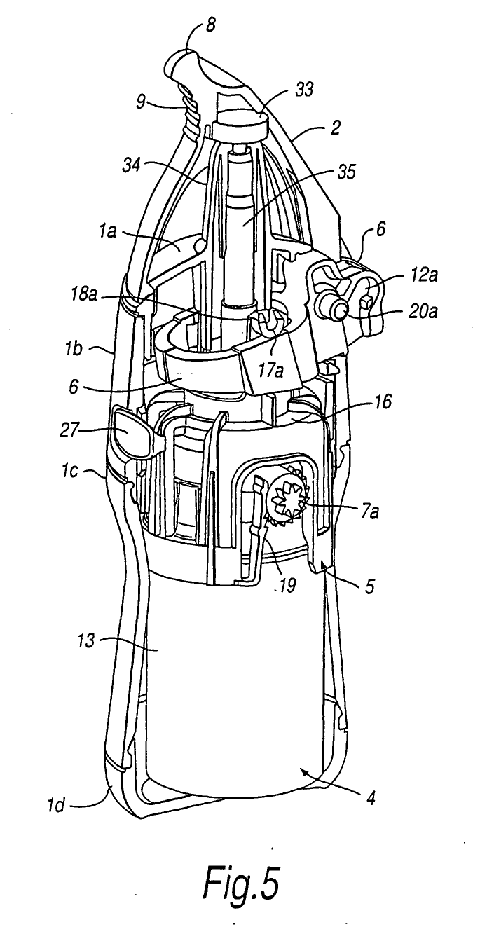

FIG. 5 is a partial sectional view of the delivery device in FIG. 3 (without the counting ring);

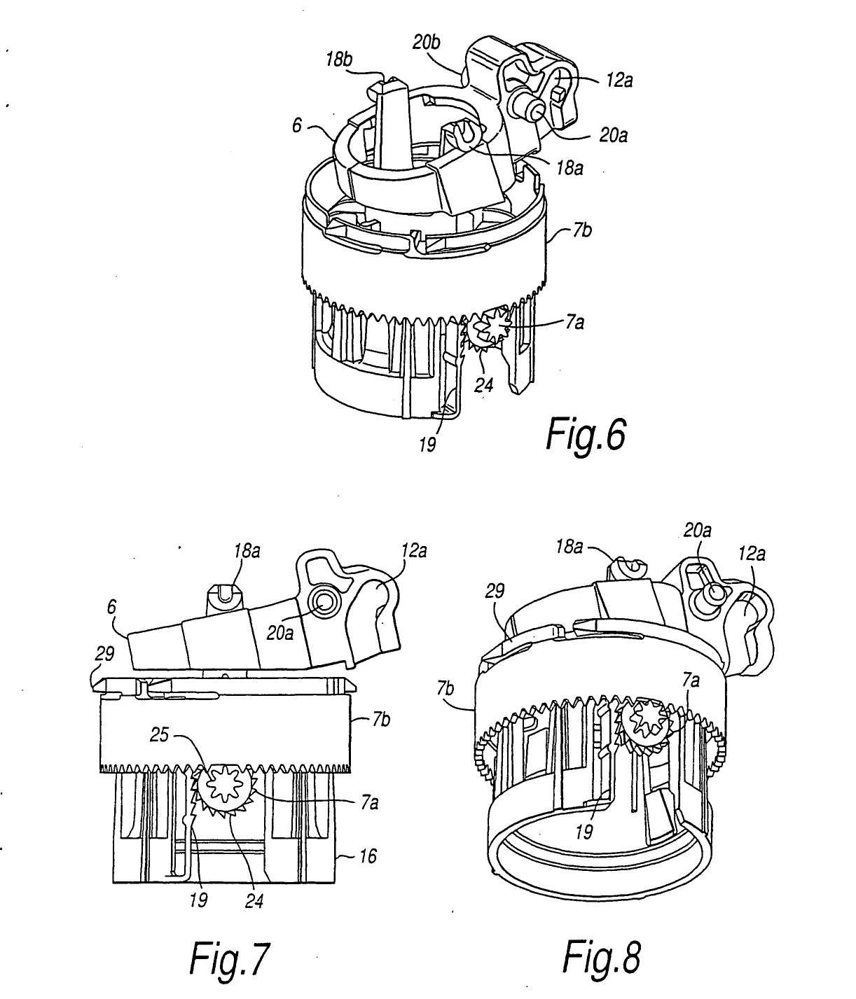

FIG. 6 is a perspective view from above of the interactive elements of the counting mechanism of the delivery device;

FIG. 7 is a side view of the elements of the delivery device in FIG. 6;

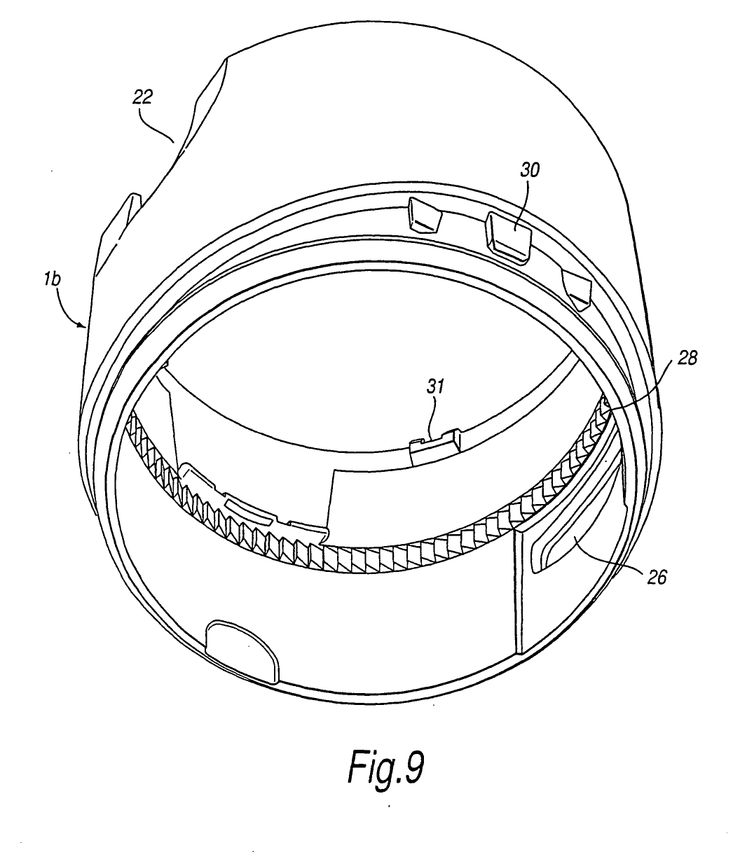

FIG. 8 is a perspective view from below of the elements of the delivery device in FIG. 6;

FIG. 9 is a view from below of the upper body portion of the housing;

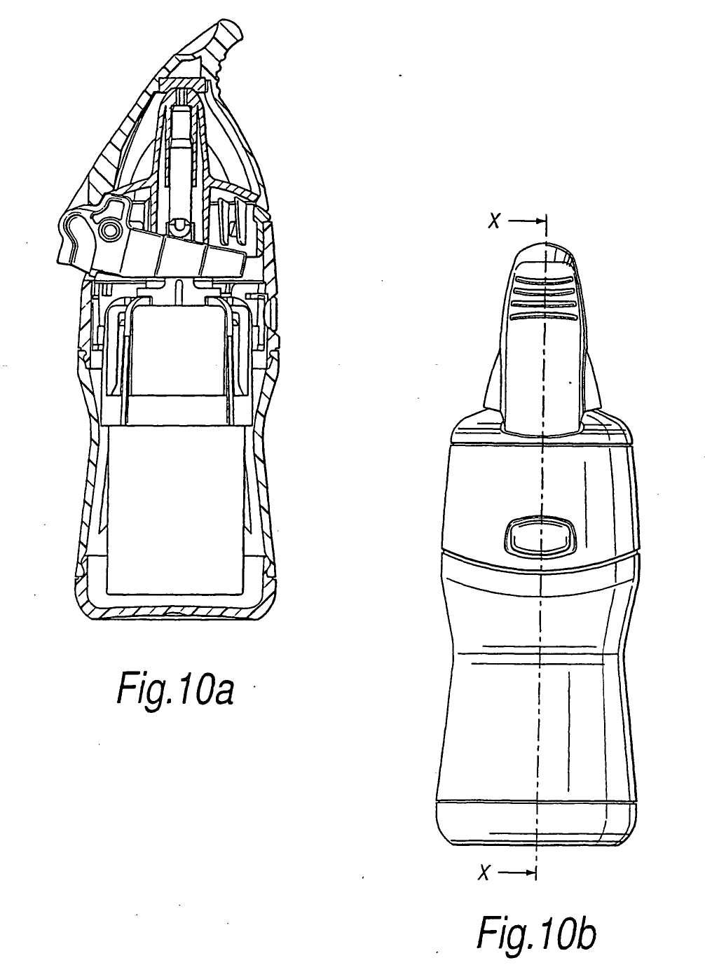

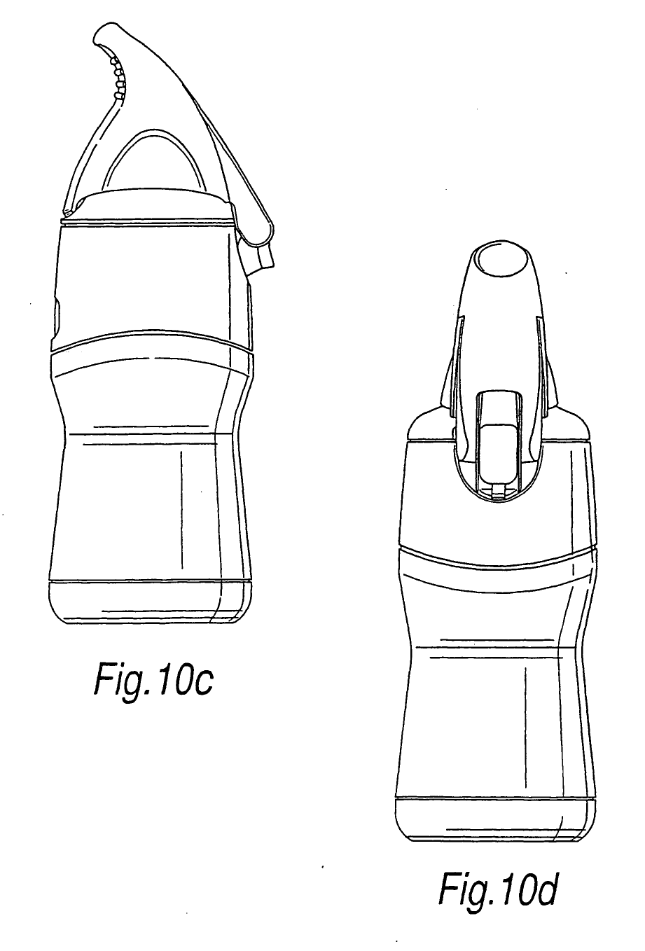

FIGS. 10a to 10d are various views of the delivery device with the cover member in the closed position;

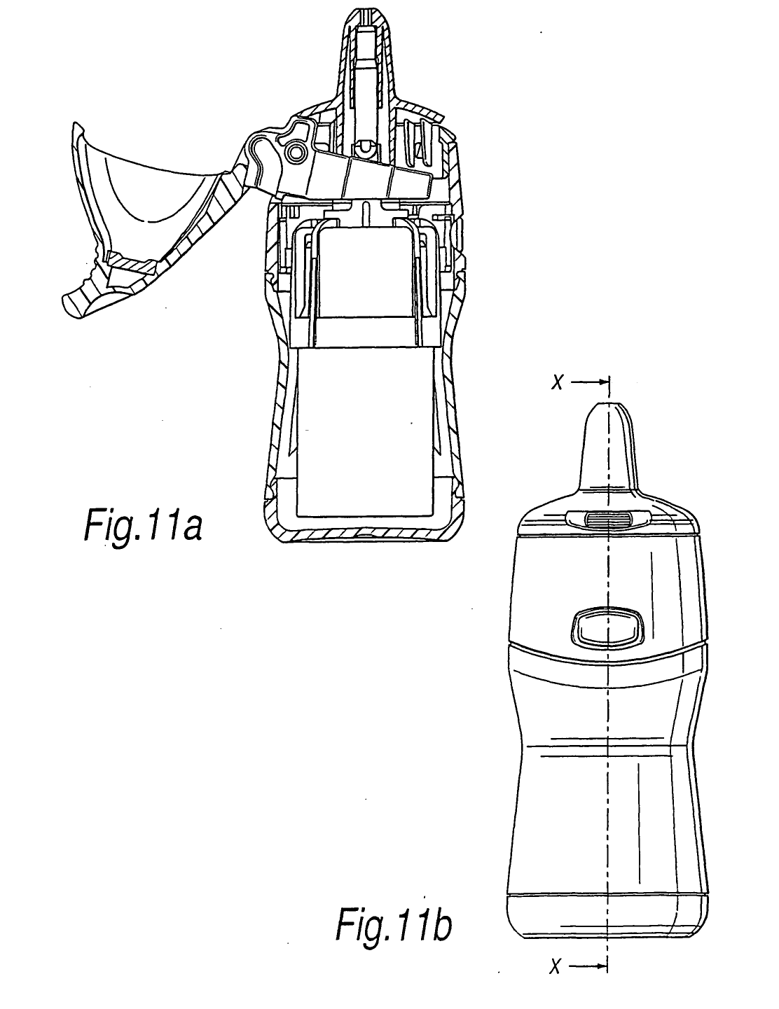

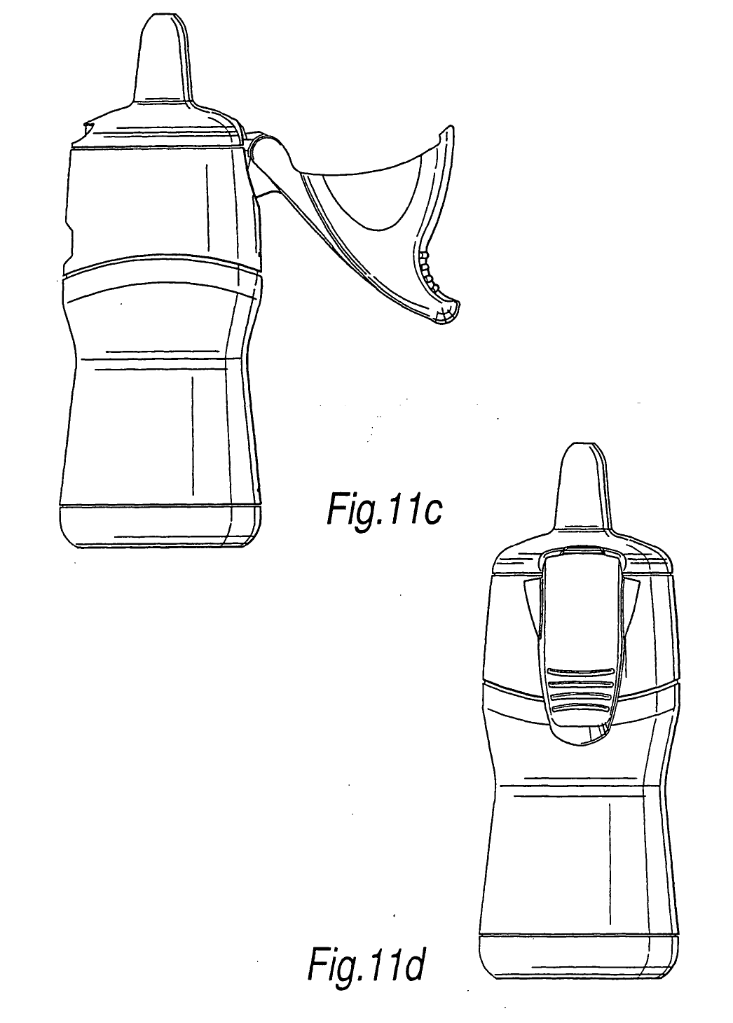

FIGS. 11a to 11d are various views of the delivery device with the cover member in the open position prior to actuation;

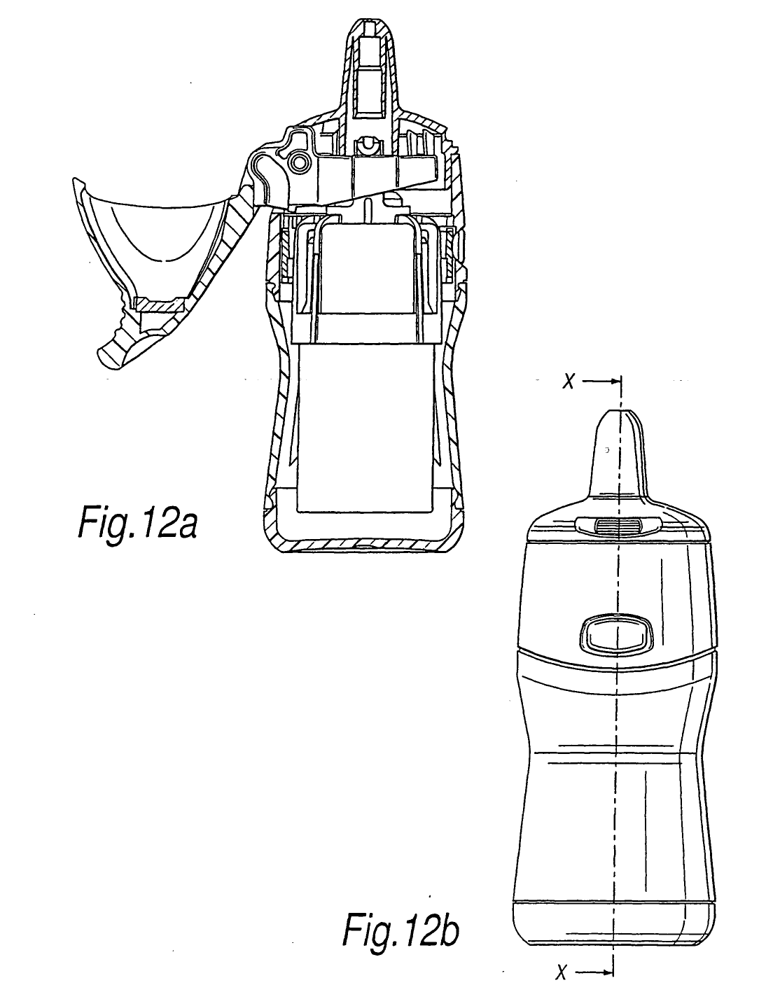

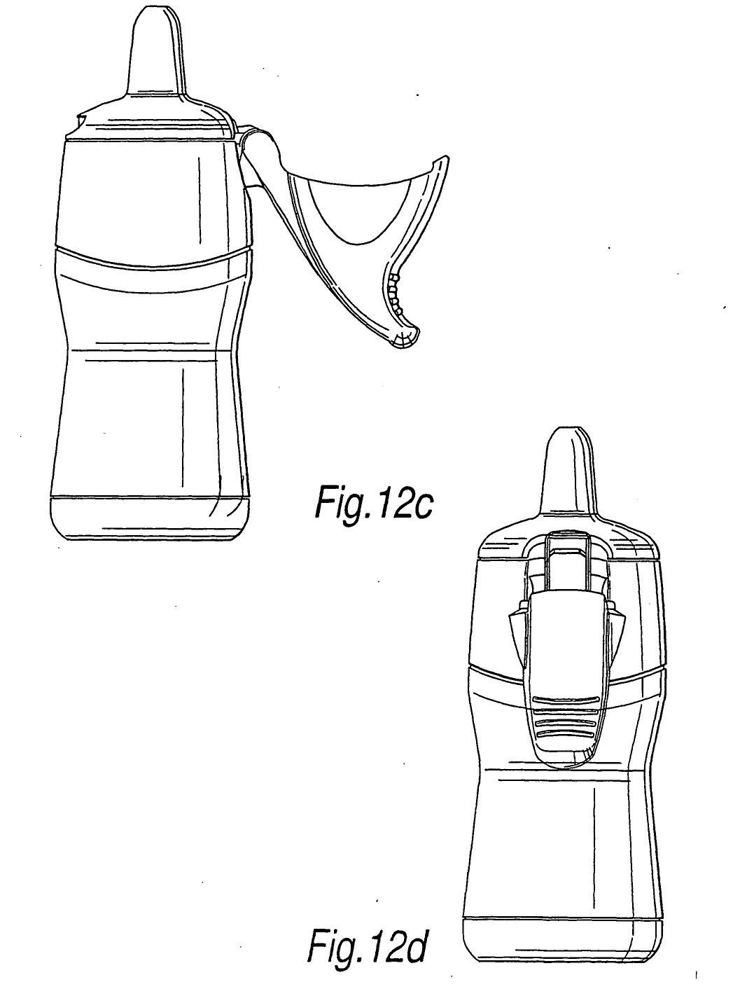

FIGS. 12a to 12d are various views of the delivery device at the point of actuation;

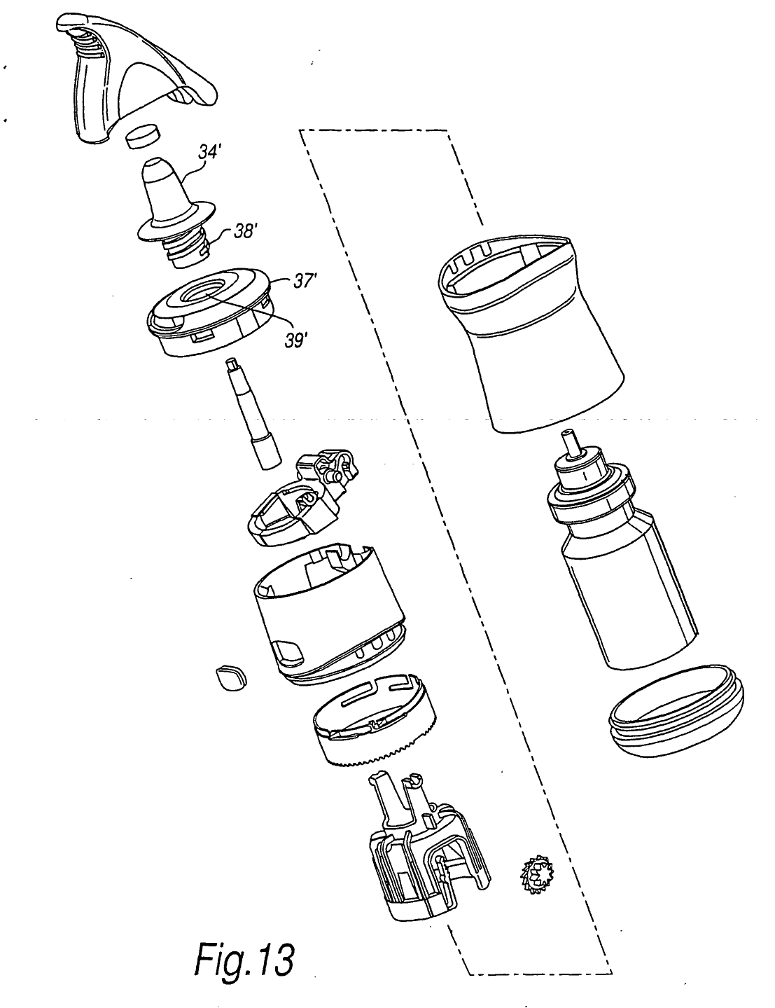

FIG. 13 is an exploded perspective view from above of a second embodiment of the delivery device;

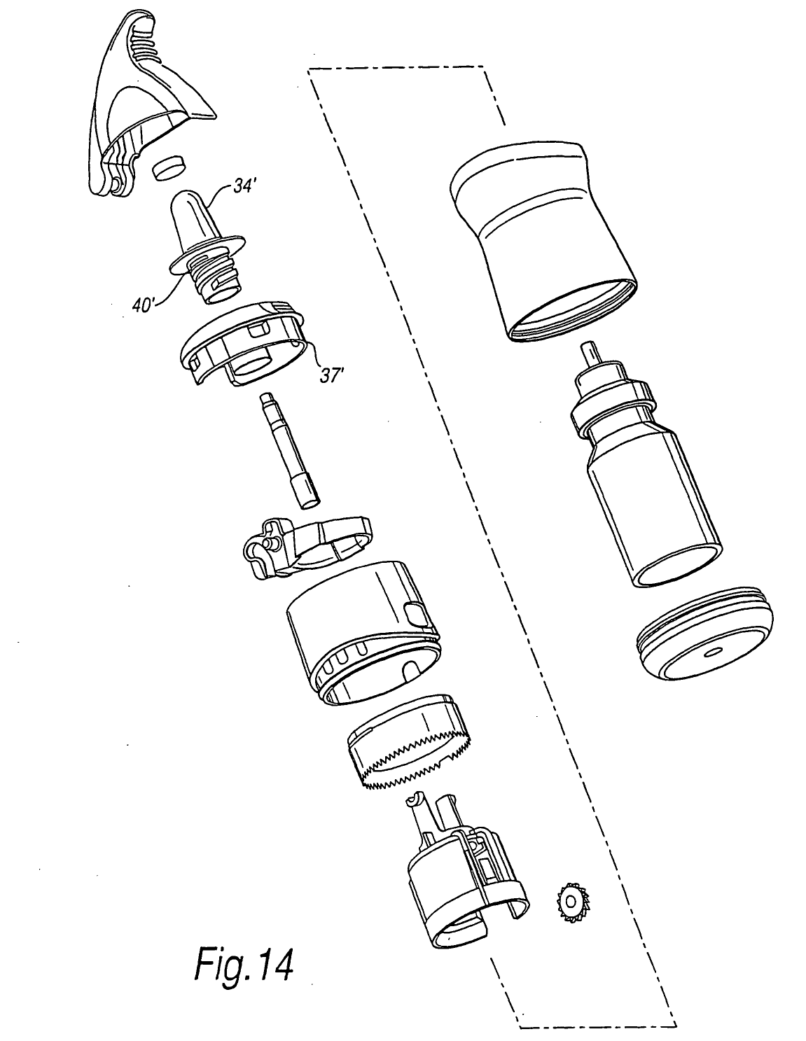

FIG. 14 is an exploded perspective view from below of the second embodiment of the delivery device; and,

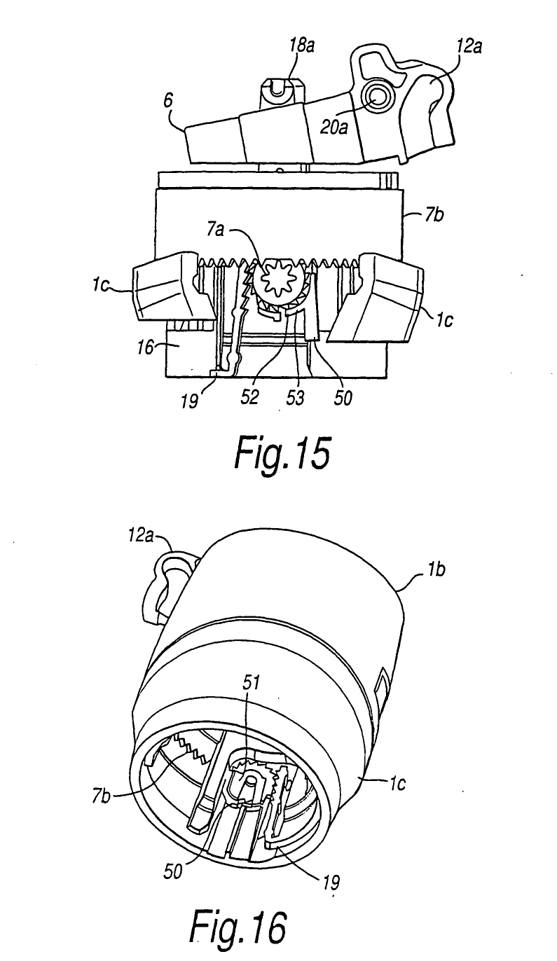

FIGS. 15 and 16 depict an alternative anti-back drive mechanism.

The delivery device depicted in FIGS. 1 and 2 comprises a housing 1, having an applicator portion 1a, an upper body portion 1b, a lower body portion 1c and a base portion 1d, an actuating member 2 and a liquid delivery assembly 3.

The liquid delivery assembly 3 comprises a liquid delivery unit 4, a carrier unit 5 which carries the liquid delivery unit 4 and a coupling member 6 which couples the movement of the actuating member 2 and the carrier unit 5.

The actuating member 2 is in the form of a cover member and is moveable relative to the housing 1 (1a, 1b, 1c, 1d) between a first position to a second position which actuates the delivery device. Although the actuating member 2 is shown as a cover which pivots on the coupling member 6, it will be appreciated that other forms of actuating member could be used, the requirement being that in the second position, the actuating member moves the carrier unit 5 as required.

The delivery device also comprises a counting mechanism 7a, 7b which indicates to the user how many doses of liquid have been delivered. This feature helps to avoid the user attempting to actuate the delivery device when empty and also serves as a reminder that a replacement delivery device should be obtained when only a few doses remain.

FIG. 2 gives a different perspective of the exploded view in FIG. 1, showing the elements from below rather than above.

FIG. 3 is a perspective view of the assembled delivery device with the cover member 2 in the first position (closed) prior to actuation by a user. FIG. 4 depicts the delivery device when the cover member 2 has been moved into the open position prior to actuation.

FIG. 5 is a partial sectional view through the delivery device in FIG. 3 with the housing 1 and cover member 2 sectioned and the internal elements non-sectioned. The counting ring 7b of the counting mechanism has been removed to enable the remaining internal elements to be seen more clearly.

The cover member 2 has a basic hollow domed shape contoured to fit over the applicator portion 1a. A tapered tip 8 with a series of gripping ridges 9 facilitates opening of the delivery device. The cover member 2 is also provided with an extension 10 which carries spaced pins 11a, 11b which sit in sockets 12a, 12b on the coupling member 6. The applicator portion 1a has a slot 13 into which a locking member 14 on the cover member 2 fits to hold the cover member 2 in position when closed. The locking member 14 should be configured such that the user has to apply little force to the tapered tip 8 to make the cover member 2 pivot into the open position.

The main elements of the liquid delivery assembly 3 are clearly visible in FIG. 5. The liquid delivery assembly 3 comprises a liquid delivery unit 4, a carrier unit 5 for the liquid delivery unit and the coupling member 6.

The liquid delivery unit 4 comprises a container 13 and a pump 14 having a tubular nozzle 15. The tubular nozzle 15 is slidable into the container 13 against the action of an internal spring (now shown) to deliver a volume of liquid.

The carrier unit 5 comprises a cage 16 constructed to sit around the pump 14 and tubular nozzle 15 of the liquid delivery unit 4. The cage 16 holds and carries the liquid delivery unit 4 during actuation. At the upper end of the cage 16 are spaced arms 17a, 17b, each of which has a hook 18a, 18b configured to rest on the coupling member 6 when the delivery device is assembled. A resilient toothed ratchet finger 19 extends from the lower end of the cage 16 and cooperates with a gear member 7a of the counting mechanism.

The coupling member 6 is in the form of a yoke which can pivot on the upper body portion 1b of the housing. Projections 20a, 20b on each side of the coupling member 6 sit in sockets 21a, 21b on each side of a lateral opening 22 in the upper body portion 1b. The applicator portion 1a is also provided with a lateral opening 23 to accommodate the pivoting movement of the coupling member 6.

The counting mechanism comprises a counting ring 7b and a gear member 7a which interact with the toothed ratchet finger 19. As is evident from FIGS. 5 to 8, any movement of the toothed ratchet finger 19 will turn the gear member 7a via the outer set of teeth 24 which in turn will result in the inner set of teeth 25 on the gear member 7a turning the counting ring 7b. The tooth pitch on the ratchet finger 19 is designed such that the gear member 7a moves the counting ring 7b forward by one tooth for each dose delivered. The gear member 7a sits in a gear pocket 49 (FIG. 1a) on the upper internal surface of the lower body portion 1c. The counting ring 7b will typically be calibrated with an indication of how many doses remain and this number will be visible through a window 26 in the upper body portion 1b. A transparent cover 27 for the window is provided to prevent the internal mechanism becoming clogged with particles of dust or dirt, for example, and to prevent tampering.

The upper body portion 1b is depicted in FIG. 9 viewed from below. On the internal surface a ring of teeth 28 is provided which will cooperate with four anti-back drive fingers 29 on the counting ring 7b. The circumferential position of the teeth 28 is arranged to have a vernier action such that only one of the four fingers 29 will be engages at any time. The delivery device is designed to lift the ratchet finger 19 upwards during actuation driving the gear member 7a in a clockwise direction (see FIG. 7). The outer set of teeth 24 on the gear member 7a then turn by one tooth in a clockwise direction as the ratchet finger 19 moves upwards by one tooth. However, after actuation when the ratchet finger 19 moves downwards on the return stroke, there will be tendency to turn the gear member 7a in an anticlockwise direction and hence a tendency to turn the counting ring 7b in a reverse direction. The anti-back drive fingers 29 are provided to prevent any reverse rotation of the counting ring 7b which would affect the counting mechanism and possibly distort the true reading of the remaining doses.

The upper body portion 1b is also provided with suitable projections 30, 31 on the open ends which serve to lock the housing portions together during assembly. Cooperating recesses or projections can also be found on the applicator portion 1a, the lower body portion 1c and the base portion 1d.

Preferably, the cover member 2 has a seal 33 which sits in the tapered tip 8 which ensures that the outlet nozzle 34 of the applicator portion 1a is suitably sealed when the cover member 2 is in the closed position. A tubular feed 35 sits above the tubular nozzle 15 of the liquid delivery unit 4 and extends into the outlet nozzle 34. The tubular feed 35 guides the dose of liquid from the tubular nozzle 15 to the outlet 36 in the outlet nozzle 34.

Whilst the seal 33 has been described with reference to the particular delivery device described and depicted in FIGS. 1 to 16, it will be appreciated that such a seal could be incorporated into a wide range of alternative delivery devices which feed a liquid to an outlet nozzle. For example, the cover member could be a snap-fit onto the outlet nozzle or could have a screw-thread. The cover member should in all cases be constructed such that the seal 33 is held with a substantially constant pressure against the outlet nozzle.

FIGS. 13 and 14 depict a second embodiment of the present invention which differs only in that the outlet nozzle 34′ is removable from the base 37′ of the applicator portion 1 for cleaning purposes. A screw thread 38′ is provided on the outlet nozzle 34′ with a correspondingly threaded socket 39′ in the base 37′. At the end of the screw thread 38′ provides a “snap-action” to indicate to the user that the outlet nozzle 34′ has been correctly replaced.

In use, a user takes the delivery device in one hand and flips the cover member 2 from the closed position into the open position. See FIGS. 10a, 10b, 10c and 10d which depict various views of the delivery device with the cover member 2 in the closed positions. FIGS. 11a, 11b, 11c and 11d depicts the delivery device after the cover member 2 has pivoted into the open position via pins 11a, 11b in sockets 12a, 12b.

The delivery device should then be positioned with the outlet nozzle 34 in a nasal cavity. The user then applies further pressure on the cover member 2 such that the coupling member 6 pivots via projections 20a, 20b in sockets 21a, 21b in the upper body portion 1b. The coupling member 6 then starts to lift the carrier unit 5 by cooperation with the hooks 18a and 18b on the arms 17a and 17b. See FIGS. 12a, 12b, 12c and 12d.

Since the liquid delivery unit 4 is carried by the carrier unit 5, the container 13, pump 14 and tubular nozzle 15 all move upwards until the tubular feed 35 forces the tubular nozzle 15 into the container 13 to actuate liquid delivery by way of pump 14.

During actuation the toothed ratchet finger 19 engages the counting mechanism as described earlier to move the counting ring 7b and display through window 26 how many doses remain.

After actuation, the user removes pressure on the cover member 2 which allows the coupling member 6 to return to the position in FIGS. 11a to 11d. The cover member 2 should then be pivoted back to the closed position until a further dose is required.

FIGS. 15 and 16 depict an alternative anti-back drive mechanism. The four anti-back drive finger 29 on the counting ring 7b have been replaced by a ratchet arm 50 which extends from the internal surface of the lower body portion 1c. The ring of the teeth 28 are no longer necessary on the internal surface of upper body portion 1b. In FIG. 15, the lower body portion 1c is only shown in part so that the locations of the carrier 16 and ratchet arm 50 are clearly visible.

The ratchet arm 50 extends into a gear pocket 51 which is located on the internal surface of the lower body portion 1c. As in the earlier embodiment described with reference to FIGS. 7, 8 and 9, the ratchet arm 50 will prevent the gear member 7a from turning in an anticlockwise direction (in FIG. 15) when ratchet finger 19 moves downwards on the return stroke. The ratchet arm 50 has two teeth 52 and 53 which allow clockwise rotation of the gear member 7a but act as a back-stop against anticlockwise rotation. The provision of two teeth 52,53 helps to reduce backlash.

FIG. 16 is a perspective view from below of the upper and lower body portions 1b, 1c with gear pocket 51, ratchet finger 19 and ratchet arm 50 visible.

1. A manually actuated delivery device for the delivery of a dose of liquid comprising a housing which includes an outlet through which liquid, in use, is delivered, an actuating member which is moveably disposed relative to the housing between a first position and a second position, the user moving the actuating member into the second position to actuate the delivery device and a liquid delivery assembly which is located at least partly within the housing, the liquid delivery assembly comprising a liquid delivery unit, a carrier unit for moving the liquid delivery unit along the longitudinal axis of the device and a coupling member which couples the movement of the actuating member and the carrier unit, characterised in that movement of the carrier unit along the longitudinal axis of the device activates a counting mechanism which indicates how many doses have been delivered, and a seal is located within the cover member to seal the outlet when the actuating member is in the first position.

2. A manually actuated delivery device as claimed in claim 1, wherein the carrier unit comprises at least one toothed ratchet finger which cooperates with a gear member of the counting mechanism.

3. A manually actuated delivery device as claimed in claim 2, wherein the gear member engages a toothed counting ring of the counting mechanism.

4. A manually actuated delivery device as claimed in claim 3, wherein the tooth pitch on the ratchet finger is constructed such that the gear member moves the toothed counting ring forward by one tooth for each dose delivered.

5. A manually actuated delivery device as claimed in claim 3, wherein anti-back drive means are provided to prevent the toothed counting ring rotating in a reverse direction.

6. A manually actuated delivery device as claimed in claim 5, wherein the anti-back drive means are located on the counting ring.

7. A manually actuated delivery device as claimed in claim 5, wherein the anti-back drive means are located on the internal surface of the housing.

8. A manually actuated delivery device as claimed in claim 7, wherein the anti-back drive means are in the form of a ratchet arm which engages the gear member.

9. A manually actuated delivery device as claimed in claim 3, wherein the gear member has an outer set of teeth which engage the toothed ratchet finger and an inner set of teeth which engage the toothed counting ring.

10. A manually actuated delivery device as claimed in claim 1, wherein the actuating member is a cover member which is moveably disposed between the first position in which the cover member at least partly covers the outlet and the second position in which the cover member acts as a lever which lifts the carrier unit.

11. (canceled)

12. A manually actuated delivery device as claimed in claim 1, wherein the housing comprises an applicator portion with an outlet nozzle, the outlet nozzle being removable to enable cleaning.

13. A manually actuated delivery device as claimed in claim 12, wherein the outlet nozzle is threaded and cooperates with a threaded socket in the applicator base portion.

14. A manually actuated delivery device as claimed in claim 13, wherein the outlet nozzle “snaps” into the final position in the threaded socket.

15. A manually actuated delivery device for delivery of a dose of liquid comprising a housing which includes an outlet through which liquid, kin use, is delivered, and a cover member which is moveable between a first position in which the outlet is covered and a second position in which the outlet is exposed characterised in that a seal is located within the cover member to seal the outlet when in the first position.

16. A manually actuated delivery device as claimed in claim 15, wherein the cover member is hinged on the housing.

17. A manually actuated delivery device as claimed in claim 15, wherein the cover member is a snap-fit on the housing.

18. A manually actuated delivery device as claimed in claim 15, wherein the cover member has a screw-thread which cooperates with a screw-thread on the housing.

19. (canceled)
